(19) United States
(12) Patent Application Publication
(10) Pub. No.: US 2023/0256193 A1
(43) Pub. Date: Aug. 17, 2023

(54) MEDICAL DEVICE

(71) Applicant: OLYMPUS MEDICAL SYSTEMS CORP., Tokyo (JP)

(72) Inventor: Kayo NOMURA, Tokyo (JP)

(73) Assignee: OLYMPUS MEDICAL SYSTEMS CORP., Tokyo (JP)

(21) Appl. No.: 18/109,508

(22) Filed: Feb. 14, 2023

Related U.S. Application Data
(60) Provisional application No. 63/310,646, filed on Feb. 16, 2022.

(51) Int. Cl. A61M 25/00; A61M 25/10

(52) U.S. Cl. CPC A61M 25/0017; A61M 25/10

(57) ABSTRACT

A medical device includes: a medicinal agent storage device connected to the catheter, a flow control device, and a package. The medicinal agent storage device includes a storage part and a second channel, the storage part is configured to store a medicinal agent, and wherein the second channel provides fluid communication between the storage part and the first channel. The package encloses the catheter, the medicinal agent storage device, and the flow control device in a sterilized state. In a first state, the flow control device is configured to prevent the medicinal agent from flowing from the storage part into the first channel. The flow control device is included in the catheter or included in the medicinal agent storage device.

### RELATED APPLICATION DATA

This application is based on and claims priority under 37 U.S.C. § 119 to U.S. Provisional Application No. 63/310,646 filed on Feb. 16, 2022, the entire contents of which are incorporated herein by reference.

### BACKGROUND

In recent years, for medical devices, such as endoscopes or catheters, used in a medical field, single-use medical devices that are discarded after being used one time have been used in order to prevent infectious disease or the like. Sterilizing treatment is applied to such medical devices before shipping, and the medical devices are sealed in a package, such as a sterilization pack, while maintaining a sterilized state.

As an examination that uses such medical devices, there is an endoscopic retrograde cholangiopancreatography (ERCP). ERCP is an examination where an endoscope is introduced from the mouth and is caused to proceed to the duodenum, then a catheter, such as a contrast tube, is inserted through a treatment instrument insertion channel of the endoscope and, thereafter, a contrast agent is injected into the bile duct or the pancreatic duct from a distal end of the endoscope via the catheter in order to examine abnormalities in the bile duct or the pancreatic duct.

For example, Japanese Patent Application Laid-Open Publication No. H9-99089 discloses a catheter that can be used for ERCP.

### SUMMARY OF THE DISCLOSURE

A medical device according to one aspect of the present disclosure includes: a medicinal agent storage device connected to the catheter, a flow control device, and a package. The medicinal agent storage device includes a storage part and a second channel, the storage part is configured to store a medicinal agent, and wherein the second channel provides fluid communication between the storage part and the first channel. The package encloses the catheter, the medicinal agent storage device, and the flow control device in a sterilized state. In a first state, the flow control device is configured to prevent the medicinal agent from flowing from the storage part into the first channel. The flow control device is included in the catheter or included in the medicinal agent storage device.

### BRIEF DESCRIPTION OF THE DRAWINGS

FIG. 1 is a diagram showing one example of an overall configuration of a medical device according to a first embodiment.

FIG. 2 is a perspective view showing one example of a configuration of a stopper.

FIG. 3 is a diagram showing a connection state of the stopper.

FIG. 4 is a diagram showing one example of a configuration of a medical device according to a modification of the first embodiment.

FIG. 5A is a diagram showing one example of a configuration of a medical device according to a second embodiment.

FIG. 5B is a cross-sectional view showing a configuration of a stopper in the second embodiment.

FIG. 5C is a front view showing the configuration of the stopper in the second embodiment.

FIG. 6A is a partial cross-sectional view showing one example of a configuration of a medical device according to a third embodiment.

FIG. 6B is a perspective view showing a configuration of a stopper in the third embodiment.

FIG. 7A is a diagram showing one example of a configuration of a medical device according to a fourth embodiment.

FIG. 7B is a perspective view showing one example of a configuration of a filter member in the fourth embodiment.

FIG. 8 is a cross-sectional view showing an arrangement example of a filter member according to a modification of the fourth embodiment.

### DETAILED DESCRIPTION

In general, a medicinal agent, such as a contrast agent (for example, meglumine sodium amidotrizoate injection) used in ERCP is drawn into a syringe from a bottle or an ampoule and, thereafter, is injected into the bile duct or the pancreatic duct by making use of a catheter. Therefore, when the medicinal agent is drawn into the syringe from the bottle or the ampoule, or when the syringe is connected to the catheter, there is a risk of bacteria, such as environmental bacteria, coming into contact with the medicinal agent.

According to embodiments described hereinafter, it is possible to provide a medical device that can reduce a risk of bacteria coming into contact with the medicinal agent.

Hereinafter, the embodiments will be described in detail with reference to drawings.

Note that the drawings based on the embodiments are schematic views, and a relationship between thicknesses and widths of respective portions, a ratio between thicknesses of the respective portions, and relative angles, for example, may differ from actual ones. The dimensional relations and the ratio may differ in some parts between the drawings.

### First Embodiment

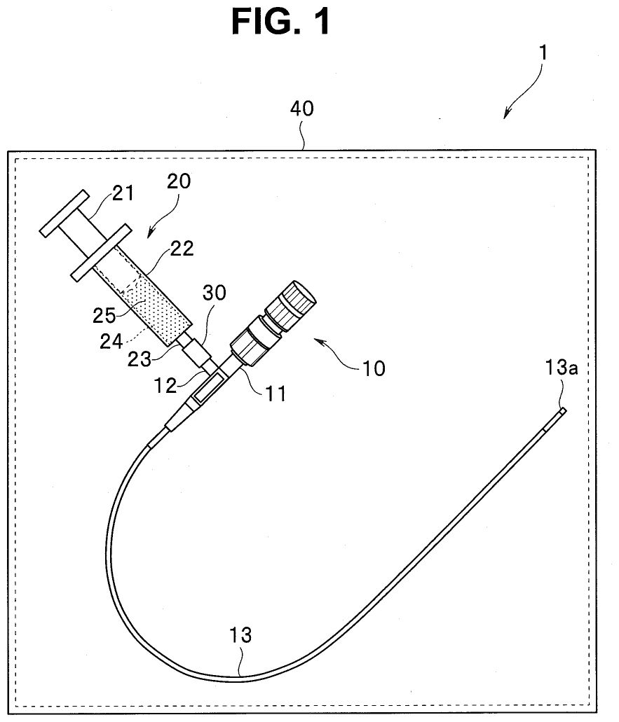

FIG. 1 is a diagram showing one example of an overall configuration of a medical device according to a first embodiment. A medical device 1 shown in FIG. 1 includes a catheter 10, a syringe (medicinal agent storage device) 20, a stopper 30, and a package 40.

The catheter 10 includes an operation portion 11, a liquid feeding pipe sleeve 12, and an insertion portion 13, an operator performing an operation, such as feeding liquid, with the operation portion 11 at a hand side, the syringe 20 being connected to the liquid feeding pipe sleeve 12 via the stopper 30, the insertion portion 13 having flexibility. The insertion portion 13 includes a distal end portion 13a. The catheter 10 has a liquid feeding channel (first channel) for feeding liquid into the operation portion 11, the liquid feeding pipe sleeve 12, and the insertion portion 13.

The distal end portion 13a of the insertion portion 13 is, for example, inserted through a treatment instrument insertion channel via a treatment instrument insertion opening provided at an operation portion of an endoscope, and is caused to protrude from a distal end opening of the insertion portion of the endoscope.

The syringe 20 being a medicinal agent storage device includes a pusher 21, an outer sleeve 22, and a conduit (second channel) 23. The outer sleeve 22 includes a storage part 24 in an inside of the outer sleeve 22, a medicinal agent 25, such as a contrast agent, being stored in the storage part 24. The conduit 23 communicates with an upstream side of the liquid feeding channel of the catheter 10. The second channel 23 provides fluid communication between the storage part 24 and the first channel. The medicinal agent storage device 20 is connected to the catheter 10. A configuration may be adopted where the conduit 23 is directly connected to the liquid feeding pipe sleeve 12 of the catheter 10. The conduit 23 forms an attachment/detachment part that is attached to or detached from the catheter 10. The medicinal agent storage device 20 can be attachable to and detachable from the catheter 10. In the description made hereinafter, a direction from the catheter 10 to the syringe 20 is referred to as an upstream direction, and a direction from the syringe 20 to the catheter 10 is referred to as a downstream direction. The medicinal agent 25 is contained in the storage part 24. When the pusher 21 is pushed with the storage part 24 store or filled with the medicinal agent 25, it is possible to inject the medicinal agent 25 into the liquid feeding channel of the catheter 10 via the conduit 23. In contrast, when the pusher 21 is pulled in a state where the pusher 21 is pushed into the outer sleeve 22, it is possible to inject the medicinal agent into the storage part 24 from the conduit 23. Note that the medicinal agent storage device that injects the medicinal agent 25 into the liquid feeding channel of the catheter 10 is not limited to the syringe 20, and may be a balloon, for example. The storage part 24 has an injection port configured to be injected the medicinal agent 25. The medicinal agent storage device 20 may be a balloon or a syringe.

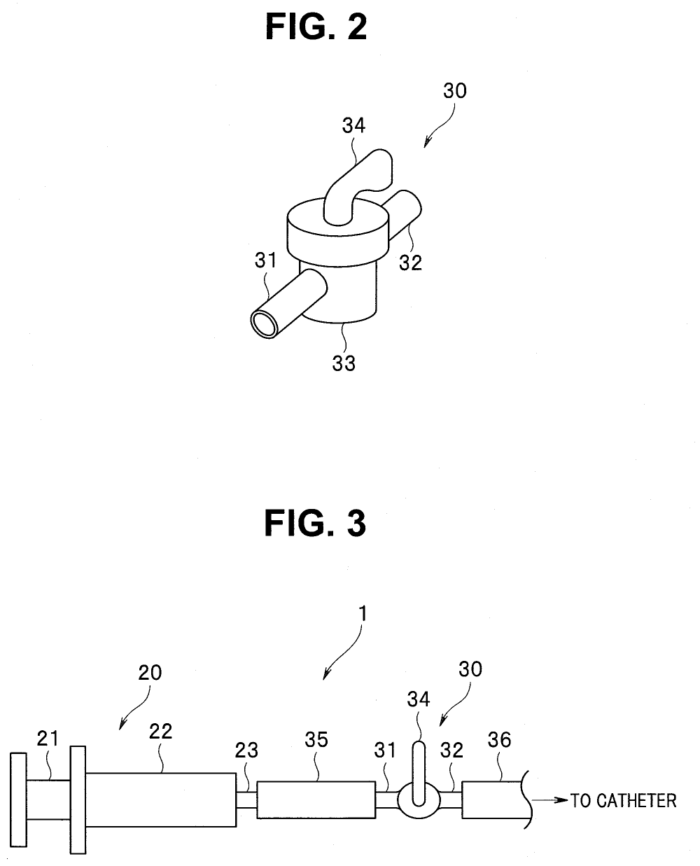

The stopper 30 is provided between the catheter 10 and the syringe 20, and prevents the medicinal agent 25 filled in the storage part 24 of the syringe 20 from flowing toward the catheter 10. A configuration of the stopper 30 is not limited to the configuration where the stopper 30 is provided between the catheter 10 and the syringe 20, and the stopper 30 may be provided to either the catheter 10 or the syringe 20. A configuration of the stopper 30 will be described with reference to FIG. 2 and subsequent drawings which will be described later. The flow control device 30 prevents the medicinal agent 25 from flowing from the storage part 24 into the second channel 23 by preventing a volume inside the storage part 24 from changing.

After the catheter 10, the syringe 20, and the stopper (flow control device) 30 are disposed in the package 40 and the package 40 is sealed, sterilizing treatment is applied. Due to such sterilizing treatment, the catheter 10, the syringe 20, the stopper 30 and the package 40 are sterilized. The package 40 encloses the catheter 10, the medicinal agent storage device 20, and the flow control device 30 in a sterilized state. The flow control device 30 may be included in the catheter 10 or included in the medicinal agent storage device 20.

The flow control device 20 is switchable between a first state and a second state. In the first state, the flow control device 30 is configured to prevent the medicinal agent 25 from flowing from the storage part 24 into the first channel. In the second state, the flow control device 20 is configured to allow the medicinal agent 25 to flow from the storage part 24 into the first channel. The flow control device 30 may include a cock or a film. The flow control device 30 may be included in the catheter 10 and be located in the first channel. The flow control device 30 may be included in the medicinal agent storage device 20 and be located in the second channel 23. The flow control device 30 may be included in the medicinal agent storage device 20 and be located on the storage part 24.

FIG. 2 is a perspective view showing one example of a configuration of the stopper, and FIG. 3 is a diagram showing a connection state of the stopper.

As shown in FIG. 2, the stopper 30 is a two-way cock including a body portion 33 and a cock 34, a first port 31 and a second port 32 being formed on the body portion 33 in a protruding manner, the cock 34 being turnably attached to the body portion 33 to open and close a flow passage in the body portion 33.

As shown in FIG. 3, the first port 31 is connected to the conduit 23 of the syringe 20 via a tube 35, and the second port 32 is connected to the catheter 10 (specifically, to the liquid feeding pipe sleeve 12 of the catheter 10) via a tube 36. When a user turns the cock 34 in a predetermined direction, the stopper 30 can bring the first port 31 and the second port 32 into either a communicating state or a cut-off state. In a case where the first port 31 and the conduit 23 have shapes that can be directly connected with each other, the tube 35 may be omitted. Further, in a case where the second port 32 and the liquid feeding pipe sleeve 12 have shapes that can be directly connected with each other, the tube 36 may be omitted.

In a state where the catheter 10, the syringe 20, and the stopper 30 are sealed in the package 40, the first port 31 and the second port 32 of the stopper 30 are in the cut-off state. Accordingly, it is possible to prevent the medicinal agent 25 in the storage part 24 of the syringe 20 from leaking into the catheter 10.

In contrast, when the package 40 is opened, and the catheter 10, the syringe 20, and the stopper 30 are taken out of the package 40 to be used, the operator turns the cock 34 in the predetermined direction. Such an operation can bring the first port 31 and the second port 32 of the stopper 30 into the communicating state. After the first port 31 and the second port 32 of the stopper 30 are brought into the communicating state, when the operator pushes on the pusher 21 of the syringe 20, it is possible to feed the medicinal agent 25 stored in the storage part 24 to the liquid feeding channel of the catheter 10.

As described above, in the medical device 1 of the present embodiment, the catheter 10, the syringe 20, and the stopper 30 to which sterilizing treatment is applied are disposed in the package 40 being a sterilization pack. Further, in the package 40, the syringe 20 in which the medicinal agent 25 is stored is connected to the liquid feeding pipe sleeve 12 of the catheter 10 via the stopper 30.

Therefore, the operator opens the package 40, and then takes out the catheter 10, the syringe 20, and the stopper 30 from the package 40. Next, the operator turns the cock 34 of the stopper 30 in the predetermined direction to bring the first port 31 and the second port 32 into the communicating state. With such operations, it is possible to immediately inject the medicinal agent 25 in the syringe 20 into the liquid feeding channel of the catheter 10. As described above, in the medical device 1 of the present embodiment, after the package 40 is opened, the medicinal agent 25 in the syringe 20 can be immediately injected into the liquid feeding channel of the catheter 10 and hence, it is possible to reduce a risk of bacteria coming into contact with the medicinal agent 25. Further, an amount of the medicinal agent 25 to be used is strictly managed depending on the application, a target part or the like. Before the first port 31 and the second port 32 are made to communicate with each other by the stopper 30, there is no possibility of the medicinal agent 25 leaking into the catheter 10 from the inside of the syringe 20. An amount of the medicinal agent 25 ejected from the syringe 20 is equal to an amount of the medicinal agent 25 injected into the catheter 10. Accordingly, it is possible to accurately know the amount of the injected medicinal agent 25 based on a scale provided on the syringe 20 and hence, it is possible to easily manage an amount of injection. In a case where the stopper 30 is not provided, there is a possibility of the medicinal agent 25 entering the catheter at the beginning and hence, it is difficult to accurately manage an amount of injection of the medicinal agent 25.

With the medical device 1 of the present embodiment, it is possible to reduce a risk of bacteria coming into contact with the medicinal agent. Further, with the medical device 1, it is possible to easily and accurately manage the amount of the medicinal agent to be injected into the body.

### (Modification)

Next, a modification of the first embodiment will be described.

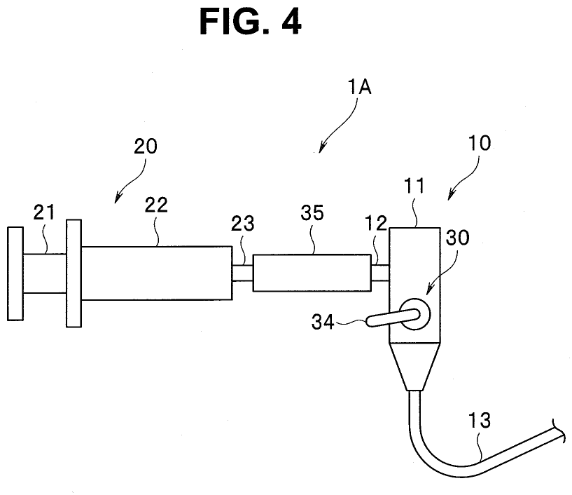

FIG. 4 is a diagram showing one example of a configuration of a medical device according to the modification of the first embodiment. In FIG. 4, components substantially equal to the corresponding components in FIG. 1 to FIG. 3 are given the same reference symbols, and the description of such components will be omitted.

As shown in FIG. 4, in a medical device 1A, the liquid feeding pipe sleeve 12 of the catheter 10 and the conduit 23 of the syringe 20 are connected with each other via the tube 35. When a configuration is adopted where the liquid feeding pipe sleeve 12 of the catheter 10 and the conduit 23 of the syringe 20 can be directly connected with each other, the tube 35 may be omitted.

In the medical device 1A of the modification of the first embodiment, the stopper 30, being a two-way cock, is provided to the operation portion 11 of the catheter 10. The first port 31 and the second port 32 of the stopper 30 are connected to a liquid feeding channel in the operation portion 11.

When the operator turns the cock 34 of the stopper 30 in the predetermined direction, it is possible to bring the first port 31 and the second port 32 into either the communicating state or the cut-off state. In the same manner as the first embodiment, in a state where the catheter 10, the syringe 20, and the stopper 30 are sealed in the package 40, the first port 31 and the second port 32 of the stopper 30 are in the cut-off state. When the operator opens the package 40 and, thereafter, turns the cock 34 of the stopper 30 in the predetermined direction to bring the first port 31 and the second port 32 into the communicating state, it is possible to immediately inject the medicinal agent 25 in the syringe 20 into the liquid feeding channel of the catheter 10.

With the medical device 1A of the modification, in the same manner as the first embodiment, it is possible to reduce a risk of bacteria coming into contact with a medicinal agent. Further, with the medical device 1A, it is possible to easily and accurately manage the amount of the medicinal agent to be injected into the body.

### Second Embodiment

Next, a second embodiment will be described.

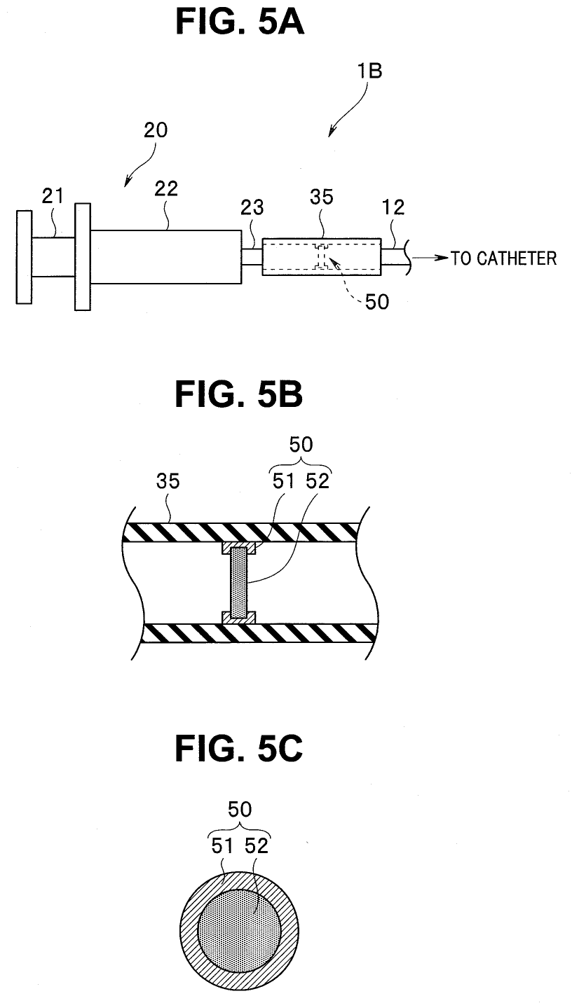

FIG. 5A is a diagram showing one example of a configuration of a medical device according to the second embodiment, FIG. 5B is a cross-sectional view showing a configuration of a stopper in the second embodiment, and FIG. 5C is a front view showing the configuration of the stopper in the second embodiment.

As shown in FIG. 5A, in a medical device 1B, the liquid feeding pipe sleeve 12 of the catheter 10 and the conduit 23 of the syringe 20 are connected with each other via the tube 35. A stopper (flow control device) 50 is disposed in the tube 35 at substantially the center in a longitudinal direction. Note that the position where the stopper 50 is disposed is not limited to substantially the center of the tube 35 in the longitudinal direction, and may be disposed at another position. The stopper 50 can have same feature and configuration as first embodiment.

As shown in FIG. 5B and FIG. 5C, the stopper 50 being a film-like member is configured to include a metal ring 51 and a film 52 made of resin, the metal ring 51 being disposed on an outer side of the stopper 50, the film 52 being disposed on an inner side of the metal ring 51. The film 52 is configured to be broken when the pusher 21 of the syringe 20 is pushed into the outer sleeve 22, so that a pressure in the storage part 24 exceeds a predetermined pressure. The film 52 is configured to break when the pressure inside the medicinal agent storage device 20 exceeds a predetermined pressure.

In a state where the catheter 10, the syringe 20, and the stopper 50 are sealed in the package 40, the film 52 of the stopper 50 can prevent the medicinal agent 25 in the storage part 24 of the syringe 20 from leaking into the catheter 10.

In contrast, when the package 40 is opened, and the catheter 10, the syringe 20, and the stopper 50 are taken out of the package 40 to be used, an operator pushes the pusher 21 of the syringe 20 into the outer sleeve 22. With such an operation, the medicinal agent 25 filled in the storage part 24 of the syringe 20 passes through the conduit 23 and breaks the film 52 of the stopper 50, and is then injected into the liquid feeding channel of the catheter 10.

With such a configuration, when the operator opens the package 40 and, thereafter, pushes the pusher 21 of the syringe 20 into the outer sleeve 22, it is possible to immediately inject the medicinal agent 25 in the syringe 20 into the liquid feeding channel of the catheter 10.

With the medical device 1B of the present embodiment, in the same manner as the first embodiment, it is possible to reduce a risk of bacteria coming into contact with a medicinal agent. Further, with the medical device 1B, it is possible to easily and accurately manage the amount of the medicinal agent to be injected into the body.

### Third Embodiment

Next, a third embodiment will be described.

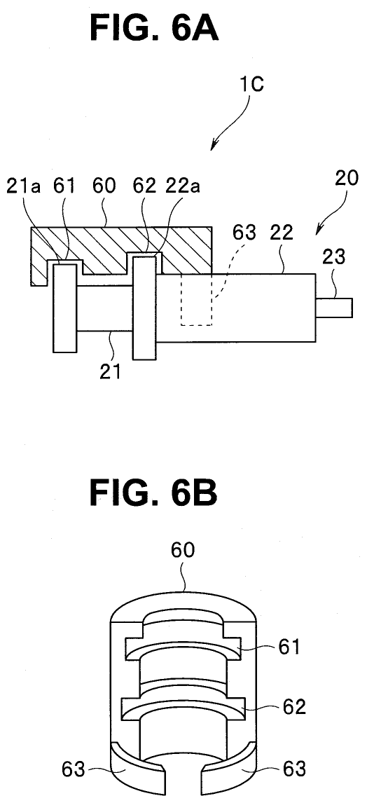

FIG. 6A is a partial cross-sectional view showing one example of a configuration of a medical device according to the third embodiment, and FIG. 6B is a perspective view showing a configuration of a stopper in the third embodiment.

As shown in FIG. 6A and FIG. 6B, in a medical device 1C, a stopper (flow control device) 60 is attached to the syringe 20. The stopper 60 is fixed to the syringe 20, and prevents the pusher 21 from being pushed on. That is to say, the stopper 60 is a guard member that prevents a change in a volume of the storage part 24. The stopper 50 can have same feature and configuration as first embodiment.

The stopper 60 includes a groove 61, a groove 62, and two locking pawls 63 having elasticity, the groove 61 engaging with a protruding portion (flange) 21a of the pusher (plunger) 21, the groove 62 engaging with a protruding portion (flange) 22a of the outer sleeve 22, the two locking pawls 63 sandwiching and locking the outer sleeve 22 of the syringe 20. The stopper 60 is fixed to the outer sleeve 22 of the syringe 20 by the two locking pawls 63. The flow control device 60 is provided on at least one of the plunger, the flange 21a, the flange 22a and outer sleeve of the syringe 20 to prevent a volume inside the storage part from changing.

In a state where the catheter 10, the syringe 20, and the stopper 60 are sealed in the package 40, the stopper 60 prevents the pusher 21 from being pushed into the outer sleeve 22 and hence, it is possible to prevent the medicinal agent 25 in the storage part 24 of the syringe 20 from leaking into the catheter 10.

In contrast, when the package 40 is opened, and the catheter 10, the syringe 20, and the stopper 50 are taken out of the package 40 to be used, an operator removes the stopper 60 from the syringe 20 and pushes the pusher 21 into the outer sleeve 22. With such operations, the medicinal agent 25 filled in the storage part 24 of the syringe 20 passes through the conduit 23, and is injected into the liquid feeding channel of the catheter 10.

With such a configuration, when the operator opens the package 40 and, thereafter, removes the stopper 60 from the syringe 20 and pushes the pusher 21 into the outer sleeve 22, it is possible to immediately inject the medicinal agent 25 in the syringe 20 into the liquid feeding channel of the catheter 10.

With the medical device 1C of the present embodiment, in the same manner as the first embodiment, it is possible to reduce a risk of bacteria coming into contact with a medicinal agent. Further, with the medical device 1C, it is possible to easily and accurately manage the amount of the medicinal agent to be injected into the body.

### Fourth Embodiment

Next, a fourth embodiment will be described.

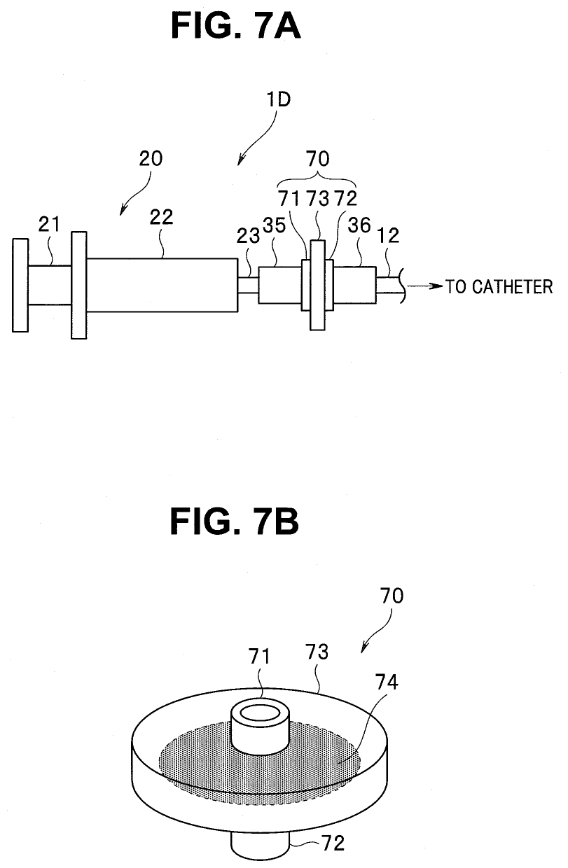

FIG. 7A is a diagram showing one example of a configuration of a medical device according to the fourth embodiment, and FIG. 7B is a perspective view showing one example of a configuration of a filter member of the fourth embodiment. The filter is located at a position downstream of the medicinal agent storage device 20, the filter is configured to filter the medicinal agent 25.

As shown in FIG. 7A, in a medical device 1D, a filter member (flow control device) 70 is provided between the liquid feeding pipe sleeve 12 of the catheter 10 and the conduit 23 of the syringe 20. Specifically, the conduit 23 of the syringe 20 and a first port 71 of the filter member 70 are connected with each other via a tube 35. Further, the liquid feeding pipe sleeve 12 of the catheter 10 and a second port 72 of the filter member 70 are connected with each other via a tube 36. That is to say, the filter member 70 is connected to the conduit 23 and the liquid feeding pipe sleeve 12 via the tubes 35 and 36. In a case where the first port 71 and the conduit 23 have shapes that can be directly connected with each other, the tube 35 may be omitted. Further, in a case where the second port 72 and the liquid feeding pipe sleeve 12 have shapes that can be directly connected with each other, the tube 36 may be omitted. The filter member 70 filters the medicinal agent 25 fed from the syringe 20, and feeds the medicinal agent 25 to the catheter 10. The filter member 70 can have same feature and configuration as first embodiment.

As shown in FIG. 7B, the filter member 70 includes a body portion 73 and a circular filter 74, the first port 71 and the second port 72 being formed on the body portion 73 in a protruding manner, the filter 74 being provided in the body portion 73. Considering that the filter 74 filters out bacteria, such as environmental bacteria, the filter 74 is made of a non-woven fabric having a pore size of 1 μm or less, and having a pore size of 0.45 μm or less, for example. The first channel has a first inner diameter, the filter has a second diameter, and the first inner diameter is smaller than the second diameter. The filter is located between the first channel and the second channel 23. The filter is located in a distal end portion of the first channel of the catheter 10.

The syringe 20 and the medicinal agent 25 in the syringe 20, which are disposed in the package 40, are already sterilized and hence, there is an extremely low risk of bacteria coming into contact with the medicinal agent. However, in a case where the medicinal agent 25 in the syringe 20 is used up, an unsterilized medicinal agent can be newly drawn into the syringe 20 from a bottle or an ampoule.

In contrast, in the configuration of the present embodiment, even in a case where an unsterilized medicinal agent is fed to the catheter 10 via the syringe 20, the medicinal agent is filtered by the filter 74 and hence, it is possible to prevent the medicinal agent that has come into contact with bacteria from being fed from the distal end portion 13a.

With the medical device 1D of the present embodiment, in the same manner as the first embodiment, it is possible to reduce a risk of bacteria coming into contact with a medicinal agent. Further, the medicinal agent cannot easily pass through the filter 74 and hence, it is possible to prevent the medicinal agent from unintentionally leaking into the catheter 10 from the syringe 20. That is to say, the filter 74 plays a role substantially equivalent to a role of the stopper 30 in the first embodiment. Accordingly, with the medical device 1D, it is possible to easily and accurately manage the amount of a medicinal agent to be injected into the body.

In the fourth embodiment, the description has been made for the configuration where the filter member 70 is disposed between the liquid feeding pipe sleeve 12 of the catheter 10 and the conduit 23 of the syringe 20 via the tubes 35 and 36. However, the filter member 70 may be disposed at another place.

### (Modification)

Next, a modification of the fourth embodiment will be described.

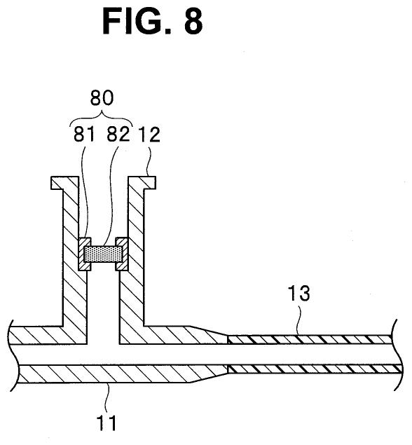

FIG. 8 is a cross-sectional view showing an arrangement example of the filter member according to the modification of the fourth embodiment.

As shown in FIG. 8, a filter member (flow control device) 80 is fixedly disposed on an inner side of (inside) the liquid feeding pipe sleeve 12 of the catheter 10. The filter member 80 has a configuration similar to the configuration of the stopper 50 shown in FIG. 5B and FIG. 5C. That is to say, the filter member 80 is configured to include a metal ring 81 and a filter 82, the metal ring 81 being disposed on an outer side of the filter member 80, the filter 82 being disposed on an inner side of the metal ring 81. The filter 82 has a configuration substantially the same as the configuration of the filter 74 shown in FIG. 7B. Considering that the filter 82 filters out bacteria, such as environmental bacteria, the filter 82 is made of a non-woven fabric having a pore size of 1 μm or less, and having a pore size of 0.45 μm or less, for example. The filter member 80 can have same feature and configuration as first embodiment.

A place where the filter member 80 is fixed is not limited to the inner side of the liquid feeding pipe sleeve 12 of the catheter 10. For example, the filter member 80 may be fixedly disposed on an inner side of the distal end portion 13a of the catheter 10, on an inner side of the insertion portion 13 of the catheter 10, or on an inner side of the conduit 23 of the syringe 20.

That is to say, it is sufficient to dispose the filter member 80 downstream of the storage part 24 of the syringe 20, the filter member 80 including the filter 82 that filters the medicinal agent 25, the storage part 24 storing the medicinal agent 25. Specifically, the filter member 80 may be disposed at any place on the conduit 23 of the syringe 20, or may be disposed at any place on a liquid feeding channel provided in the catheter 10.

The present disclosure is not limited to the above-mentioned embodiments, and various modifications and alterations are conceivable without departing from the gist of the present disclosure.

Example 1. A medical device comprising:
a catheter having a liquid feeding channel;
a medicinal agent storage device connected to the catheter and including a storage part configured to store a medicinal agent and a conduit configured to allow for communication between the liquid feeding channel and the storage part;
a stopper connected to the medicinal agent storage device and/or the catheter, and configured to prevent the medicinal agent from flowing into the catheter; and
a package sealed in a state where the catheter, the medicinal agent storage device, and the stopper are disposed in the package.
   Example 2. The medical device according to Example 1, wherein
the stopper is provided to the conduit.
   Example 3. The medical device according to Example 1, wherein
the stopper is a film-like member that is broken in a case where a pressure in the medicinal agent storage device exceeds a predetermined pressure.
   Example 4. The medical device according to Example 3, wherein
the film-like member is a film made of resin.
   Example 5. The medical device according to Example 1, wherein
the stopper is provided to the storage part.
   Example 6. The medical device according to Example 1, wherein
the stopper is a guard member that prevents a change in a volume of the storage part.
   Example 7. The medical device according to Example 1, wherein
the medicinal agent is stored in the storage part.
   Example 8. The medical device according to Example 7, wherein
the medicinal agent storage device is attachable to or detachable from the catheter.
   Example 9. The medical device according to Example 1, further comprising
a filter at a position downstream of the medicinal agent storage device, the filter being configured to filter the medicinal agent.
   Example 10. The medical device according to Example 9, wherein
the filter is disposed in the catheter at a connection part between the catheter and the medicinal agent storage device.
   Example 11. The medical device according to Example 9, wherein
the filter is disposed at a distal end portion of the catheter.
   Example 12. The medical device according to Example 8, wherein
the conduit forms an attachment/detachment part that is attached to or detached from the catheter, and the medicinal agent is injectable into the storage part from the attachment/detachment part.
   Example 13. The medical device according to Example 1, wherein
the medicinal agent storage device is a balloon.
   Example 14. The medical device according to Example 1, wherein
the medicinal agent storage device is a syringe.
   Example 15. A medical device comprising:
a catheter having a liquid feeding channel;

What is claimed is:

1. A medical device, comprising:
a catheter having a first channel; and
a medicinal agent storage device connected to the catheter, wherein the medicinal agent storage device includes a storage part and a second channel, wherein the storage part is configured to store a medicinal agent, and wherein the second channel provides fluid communication between the storage part and the first channel;
a flow control device; and
a package enclosing the catheter, the medicinal agent storage device, and the flow control device in a sterilized state,
wherein, in a first state, the flow control device is configured to prevent the medicinal agent from flowing from the storage part into the first channel, and
wherein the flow control device is included in the catheter or included in the medicinal agent storage device.

2. The medical device according to claim 1, wherein the flow control device is switchable between the first state and a second state, and
wherein, in the second state, the flow control device is configured to allow the medicinal agent to flow from the storage part into the first channel.

3. The medical device according to claim 2, wherein the flow control device is included in the catheter and is located in the first channel.

4. The medical device according to claim 3, wherein the flow control device includes a cock or a film.

5. The medical device according to claim 2, wherein the flow control device is included in the medicinal agent storage device and is located in the second channel.

6. The medical device according to claim 5, wherein the flow control device includes a cock or a film.

7. The medical device according to claim 1, wherein the flow control device is a film that is configured to be broken when a pressure inside the medicinal agent storage device exceeds a predetermined pressure.

8. The medical device according to claim 7, wherein the film made of resin.

9. The medical device according to claim 1, wherein the flow control device is included in the medicinal agent storage device and is located on the storage part.

10. The medical device according to claim 9, wherein the flow control device prevents the medicinal agent from flowing from the storage part into the second channel by preventing a volume inside the storage part from changing.

11. The medical device according to claim 1, further comprising the medicinal agent, wherein the medicinal agent is contained in the storage part.

12. The medical device according to claim 1, wherein the medicinal agent storage device is attachable to and detachable from the catheter.

13. The medical device according to claim 1, further comprising a filter located at a position downstream of the medicinal agent storage device, wherein the filter is configured to filter the medicinal agent.

14. The medical device according to claim 13, wherein the first channel has a first inner diameter,
wherein the filter has a second diameter, and
wherein the first inner diameter is smaller than the second diameter.

15. The medical device according to claim 13, wherein the filter is located between the first channel and the second channel.

16. The medical device according to claim 13, wherein the filter is located in a distal end portion of the first channel of the catheter.

17. The medical device according to claim 1, wherein the storage part has an injection port, and
wherein the medicinal agent is configured to be injected to the storage part via the injection port.

18. The medical device according to claim 1, wherein the medicinal agent storage device is a balloon.

19. The medical device according to claim 1, wherein the medicinal agent storage device is a syringe.

20. The medical device according to claim 19, wherein the flow control device is provided on a flange of the syringe to prevent a volume inside the storage part from changing.
